(19) United States
(12) Patent Application Publication
(10) Pub. No.: US 2006/0080746 A1
(43) Pub. Date: Apr. 13, 2006

(54) Methods of embryo transfer

(75) Inventors: Scott K. Davis, Bertram, TX (US); Shawn Walker, Austin, TX (US); Irina Polejaeva, Austin, TX (US)

Correspondence Address: MORRISON & FOERSTER LLP, 425 MARKET STREET, SAN FRANCISCO, CA, 94105-2482, US

(73) Assignee: ViaGen, Inc., Austin, TX (US)

(21) Appl. No.: 11/238,297

(22) Filed: Sep. 28, 2005

Related U.S. Application Data
(60) Provisional application No. 60/614,130, filed on Sep. 28, 2004.

(51) Int. Cl. A01K 67/027

(52) U.S. Cl. 800/17

(57) ABSTRACT

Methods for the efficient production of cloned porcine fetuses/piglets following the production of cloned embryos, including culture of the embryos for extended periods prior to transfer of the embryos into the uterus of the recipient. Transfer can be accomplished surgically or through less-invasive laparoscopic or non-surgical transfer.

### RELATED APPLICATIONS

This application claims the benefit of U.S. Provisional Applications No. 60/614,130 filed Sep. 28, 2004, which is incorporated by reference herein in its entirety.

### FIELD OF THE INVENTION

The present invention relates to the fields of embryology and reproductive biology.

### BACKGROUND OF THE INVENTION

Somatic cell nuclear transfer and chromatin transfer allow for the production of cloned offspring (animals genetically identical to that of the cell donor). The ability to produce clones has great value to the agricultural and biomedical industries. In particular the ability to produced clone pigs has great value to both the agricultural and biomedical fields.

From an agricultural standpoint cloning allows for the reproduction of elite animals based on predetermined genetic traits. It allows for an increase in selection intensity and a decrease in the heterogeneity of the offspring. Additionally cloning allows for the resurrection of lost genetics and allows for an increase in biosecurity. Cloning also offers the ability to cryo-bank elite genetics in preparation for a possible disease outbreak, offering insurance against bioterrorism.

In the biomedical field cloning provides a method for the rapid and efficient production of genetically modified animals as disease models. Work is progressing to generate genetically modified animals that will be immune compatible for xeno-transplantation of organs and tissues into humans. In addition, genetically modified animals are being produced by cloning to serve as bioreactors for the production of biopharmaceuticals.

The inability to successfully and efficiently culture, transport and transfer cloned embryos has limited the broad use of porcine cloning. Until now the utilization of cloned pigs in either the biomedical or agricultural industries has been limited to the select few individuals possessing all components for the production of cloned pigs; such as the equipment necessary for the cloning process (i.e. micromanipulators, stereoscopes, incubators, cell culture hood, BTX machine, inverted microscope, personal trained in cell culture and nuclear transfer), animal handling facilities (i.e. barn, large number of recipients animals, animal care staff) and surgical facilities (i.e. sterile room, anesthesia machine, surgical table, all drugs and equipment necessary for surgery, trained surgical staff). In total to fully equip a lab capable of producing cloned pig would cost in excess of one million dollars. Thus there is a need for less expensive non-surgical or minimally invasive transfer methods and for methods whereby embryos may be shipped to remote service facilities that may perform the transfer.

An additional problem with current porcine cloning protocols is the need for surgical implantation and the associated difficulties and problems. Current surgical techniques for transfer are quite labor intensive, involving a high risk of infection and only allow for the use of the recipient animal once due to adhesions. A less invasive laparoscopic technique or non-surgical transfer would allow for reduced risk of infection, quicker recovery time of recipients and the repeated use of recipients for embryo transfers. The ability to successfully culture, transport and transfer cloned embryos at an off site location and/or using minimally invasive or non-surgical techniques would allow for the broader use of the technology at a reasonable price.

Current porcine cloning protocols also typically require the direct transfer of the newly cloned embryo to the recipient's oviduct. Oviductal transfer carries the risk of trauma and damage to the recipient, including compromising the recipient's reproductive system which can impact the successful development of the cloned embryo to term. Delaying transfer would allow embryos to develop further and allow for transfer further down the reproductive tract, i.e., in the uterine horn or the uterus itself. Such transfers would be more amenable to minimally invasive or non-surgical techniques; however, reports have long suggested that prolonged culture of embryos prior to transfer leads to lower corresponding viabilities and efficiencies. For example, there are numerous reports that porcine embryonic development in vitro is retarded and results in fewer cell numbers compared to those embryos produced in vivo. In one study, total cell numbers of in vivo cultured embryos were twice that of those cultured in NCSU-23, the most commonly used porcine embryo culture medium, and had a higher ICM:trophectoderm (TE) ratio. (Machaty Z, et al. Biol Reprod 59, 451-455 (1998)). In addition, development to conceptuses was also almost 50% higher for in vivo cultured embryos than for in vitro embryos. In addition, when in vitro produced 1-cell embryos (6 h after IVF) were transferred to recipient animals, cultured in vivo for 5-days, and then flushed, the blastocysts had more than 100 nuclei. However, when the embryos where cultured in vitro instead of in vivo, development was delayed one day and the inner cell mass was poorly developed, demonstrating that low cell numbers associated with in vitro produced porcine eggs are at least partly attributable to in vitro culture conditions. Even when in vivo produced day 5 and day 6 porcine embryos were cultured for 1-3 days in Ham's F-12, or in immature mouse oviducts, morphology of the resulting blastocysts was similar to those developed in vivo but cell numbers were lower. (Papaioannou V. E., et al. Development 102: 793-803 (1988)). Thus extended in vitro culture of porcine embryos has been considered detrimental and not conducive to efficient and economical production of cloned pigs.

A further problem with current porcine cloning protocols is the preferred recipients. Cloned embryos typically are transplanted into young virgin females, i.e., gilts, because they are smaller and have less accumulated fat to navigate in performing the surgical transfer. However, the youth of such recipients makes them less fertile and their lack of experience makes them poor mothers. Furthermore, they can only be used once. Thus, there is a need for non-surgical or laparoscopic transfer so that sows that are at their peak reproductive potential may be used. In addition, sows may be selected with proven maternal abilities.

To date over 12 publications discuss the successful production of cloned pigs. In 10 of 12 of these publications, the cloned embryo was directly transferred by surgical implantation in the recipient's oviduct. (See, e.g, Bondioli, K. et al., Molecular Reproduction and Development 60, 198-195 (2001); Dai, Y. et al, Nature Biotechnology 20, 251-255 (2002); De Sousa, P. A. et al., Biology of Reproduction 66, 642-650 (2002); Lai, L et al., Science 295, 1089-1091 (2002); Lee, J.-W. et al., Biology of Reproduction 69, 995-1001 (2003); Onishi, A. et al., Science 289, 118-1190 (2000); Polejaeva, I. A. et al., Nature 407, 86-90 (2000); Ramsoondar, J. J. et al., Biology of Reproduction 69, 437-445 (2003); Walker, S. C. et al., Cloning and Stem Cells 4(2), 105-112 (2002); and Yin, X. J. et al., Biology of Reproduction 67, 442-446 (2002), each of which is incorporated herein in its entirety). Only two of those publications disclose culturing manipulated embryos for more than 48 hrs. (Boquest, A. C. et al., Biology of Reproduction 66, 1283-1287 (2002) and Betthauser, J. et al., Nature Biotechnology 18, 1055-1059 (2000), each incorporated herein by reference in its entirety.) Extended culture time has been avoided due to the huge loss in development following in vitro culture, as noted above. Currently 76 hours is the longest published culture period for cloned porcine embryos prior to a transfer which produced live offspring (Betthauser, J. et al., Nature Biotechnology 18, 1055-1059 (2000)). In this particular publication 23 recipients received cloned embryos by surgical transfer of which 7 became pregnant and two farrowed giving rise to only 4 piglets. While these individuals were successful in producing live animals following transfer of 3 day old cultured embryos, their success rate per recipient female was extremely low at <10% with the largest litter being only two piglets. Thus there is a need for transfer methods that have a higher efficiency while allowing longer culture times.

The ability to efficiently and successfully culture porcine embryos produced by nuclear transfer for longer than 76 hrs and transfer embryos by a less invasive laparoscopic or non-surgical technique would enable a broader utilization of this technology.

### SUMMARY

The present invention addresses these long felt needs by providing both a method for delayed transfer and for less invasive laparoscopic or non-surgical transfer of embryos, which may be practiced separately or in combination. Suprisingly, the present inventions provides for efficient methods of porcine cloning with culturing and delayed transfer of cloned pig embryos. In a preferred embodiment, the invention describes methods for the efficient production of cloned porcine fetuses/piglets following the production of cloned embryos, including culture of said embryos until the 8-cell stage, the 16-cell stage or even later and the transfer of the embryos into the uterus of the recipient, including either laparoscopically or non-surgically.

The present invention of culturing the embryo in vitro before transfer into a recipient and laparoscopic or non-surgical transfer may be practiced in many variations. Preferred variations are described more fully in this specification. In certain embodiments, the embryo may be from any mammal and in preferred embodiments is porcine. In various embodiments, the embryo may be produced by any means, preferred embodiments include natural or artificial insemination, in vitro fertilization, and more preferred by cloning. In certain embodiments, the embryo may be transgenic or non-transgenic. In some embodiments, the embryo is transferred when it is at least at the 2-cell stage, at least at the 4-cell stage, at least at the 8-cell stage, at least at the 16-cell stage, at least at the morula stage, at least at the blastocyst stage, at least at the expanding blastocyst stage, at least at the hatching blastocyst stage, or at least at the blastula stage. In other embodiments, the embryo is transferred when it has been cultured in vitro for at least 18 hours, at least 24 hours, at least 48 hours, at least 72 hours, at least 76 hours, at least 80 hours, at least 84 hours, at least 90 hours, at least 96 hours, at least 102 hours, at least 108 hours, at least 114 hours, at least five days, at least five and one-half days, at least six days, at least six and one-half days, at least seven days, at least seven and one-half days, at least eight days, at least eight and one-half days, or at least nine days after activation or fertilization. In a preferred embodiment, the embryos are cultured in a media such as PZM or NCSU at a temperature range of 36° C. to 40° C. under humid atmosphere containing 3.5% to 6.5% CO₂ with any appropriate range of O₂, more preferably 38.5° C. in 5% CO₂:5% O₂. In another embodiment, the embryo may be stored in any atmosphere where the media is under oil to prevent evaporation.

In various embodiments, the transfer can be accomplished by surgical or non-surgical methods or by minimally invasive methods, i.e., laparoscopic methods. In preferred embodiments, the site of transfer is the uterus, most preferably, the tip, middle or base of the uterine horn, or in the uterus body itself.

In various embodiments, the efficiency of transfer and live birth is at least one and one-half times, at least two times, at least three times, at least four times, or at least five times more efficient than existing techniques. Alternatively the efficiency of transfer and live birth may be expressed as at least 15%, at least 20%, at least 30%, at least 35%, or at least 40% live births per transfer for the method claimed. Alternatively, the efficiency of transfer and live birth may be expressed in terms of the recipient farrowing rate (i.e., the % of recipients that become pregnant and go to term) and/or the average litter size per farrowing recipient. In preferred embodiments, the recipient farrowing rate is at least 30%, at least 40%, at least 50%, at least 60%, at least 70% or at least 80%. The average litter size for each farrowing recipient is at least 4 piglets, at least 5 piglets or at least 6 piglets. In various embodiments, the recipient animal may be a gilt (young virgin female) or, more preferably, a sow in its peak reproductive age or, even more preferably, a sow of proven maternal abilities.

### DETAILED DESCRIPTION OF THE PREFERRED EMBODIMENTS

The present invention is directed to both a method for delayed transfer and for less invasive laparoscopic or non-surgical transfer of embryos, which may be practiced separately or in combination. In a preferred embodiment, the invention describes methods for the efficient production of cloned porcine fetuses/piglets following the production of cloned embryos, including culture of said embryos until the morula stage or greater and transfer of the embryos into the uterus of the recipient either laparoscopically or non-surgically.

With the method of the present invention, pregnancy to term rates as high as 80% of recipients transferred with some donor cell lines and an average of 43% across multiple donor cell lines have been achieved—three times that which has previously been reported. Additionally, the litter sizes in the examples described herein have been as high as eight piglets even with an additional two days of culture, with an average litter size of at least four piglets. Published results conducted surgical transfers without commercial shipping or other transport of the cloned embryo to separate facilities, still limiting porcine cloning to those individuals close by a nuclear transfer laboratory and possessing a surgical facility. In methods of the present invention, cloned embryos produced in a central location can be cultured for up to 4-5 days or more and shipped commercially overnight to a predetermined location where the embryos would then be transferred laparoscopically or non-surgically into a customer's own recipient animals. Further, because the embryos have cultured for such a period of time, and thus are further developed, the transfers can be uterine transfers, e.g., either in the uterine horn or the uterine body, rather than oviductal transfers.

Source of the Embryos

Embryos for transplantation may be obtained from any source available to one of skill in the art. In one embodiment, the embryos may be obtained by in vitro fertilization. In a preferred embodiment, the embryos may be generated by transfer of the nuclear genetic material of a donor cell to a recipient cell. In addition, the embryos may be transgenic or non-transgenic.

Oocytes for in vitro fertilization or for use as recipients in cloning may be obtained from any source available to one of skill in the art. By way of example, oocytes may be collected in vivo by isolation from an animal a certain number of hours after the animal exhibits characteristics that is associated with estrus or following injection of exogenous gonadatrophins known to induce ovulation. Those of skill in the art would have no difficulty inducing or otherwise identifying when an animal is in estrus, as described for example in references disclosed herein. See, e.g., Gordon, 1977, “Embryo transfer and associated techniques in pigs (Gordon, ed.),” CAB International, Wallingford UK, pp. 60-76 and Kojima, 1998, “Embryo transfer,” Manual of pig embryo transfer procedures, National Livestock Breeding Center, Japanese Society for Development of Swine Technology, pp. 7-21, each of which is incorporated herein by reference in its entirety including all figures, tables, and drawings.

In addition, the oocytes may be collected from an animal not in estrous and then matured by culturing the oocytes in vitro using standard techniques known to one of skill. Such oocytes can be isolated from either oviducts and/or ovaries of live animals by a variety of methods including oviductal recovery procedures or transvaginal oocyte recovery procedures well known in the art. Furthermore, oocytes can be isolated from deceased animals. For example, ovaries can be obtained from abattoirs and oocytes can be aspirated from these ovaries. The oocytes can also be isolated from the ovaries of a recently sacrificed animal or when the ovary has been frozen and/or thawed. Oocytes obtained by the above methods and any other method available to one of skill in the art may be used to generate embryos by methods such as in vitro fertilization and cloning by transfer of the nuclear genetic material from a donor cell by nuclear transfer or by chromatin transfer for example. In vitro fertilization may be effected by any method available to one of skill in the art.

The transferred embryos may be transgenic or non-transgenic. Transgenic embryos may be generated by a number of means and may include embryos with novel genetic material introduced, genetic material deleted or “knocked-out,” or altered genetic material such as point mutations. Techniques for molecular biology for the manipulation of nucleic acids are well known in the art and include methods and tools for insertion, deletion, and mutation of nuclear and non-nuclear genetic material of mammalian cells. See by way of example, Molecular Cloning, a Laboratory Manual, 2 nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos”; WO 98/16630, Piedrahita & Bazer, published Apr. 23, 1998, “Methods for the Generation of Primordial Germ Cells and Transgenic Animal Species,” each of which is incorporated herein by reference in its entirety. Such methods include transfecting cells with foreign DNA fragments and designing of the foreign nucleic acid fragments such that they effect insertion, deletion, and/or mutation of the target genetic material.

Such methods may be used to generate transgenic cells for use as donors of nuclear genetic material in generating cloned animals or to alter the embryo directly. Transgenic donor cells may be generated in a variety of manners. For example, transgenic cells can be isolated from a transgenic animal. Examples of transgenic porcine animals are well known in the art. Materials and methods for introducing nucleic acids into cells in culture thereby converting them into transgenic cells are well known in the art, as described previously.

Further examples of methods for modifying target genetic material in a cell by insertion, deletion, and/or mutation include retroviral vectors, artificial chromosome, gene insertion, including random insertion with tissue specific promoters and homologous recombination, gene targeting, transposable elements, and/or any other method for introducing foreign nucleic acids. Additional techniques are well known in the art for deleting nucleic acid sequences from a genome, and/or altering genetic material within a cell. Examples of techniques for altering nucleic acid sequences are site-directed mutagenesis and polymerase chain reaction procedures.

Cloned embryos may be generated by any method available to one of skill in the art. Cloned embryos are generated by transfer of the nuclear genetic material of a donor cell into a recipient cell that is capable of regenerating the animal. The recipient cell is typically an oocyte, a fertilized egg, or a cell in an early stage embryo. Numerous methods for transfer of the nuclear genetic material are known. Examples may be found for example in U.S. Pat. No. 6,235,969, U.S. Pat. No. 6,700,037, U.S. Pat. No. 6,252,243, U.S. Pat. No. 6,147,276, U.S. Pat. No. 6,781,030, and U.S. Pat. App. Pub. No. 20030046722, each of which is hereby incorporated by reference in their entirety, with special emphasis on the techniques of cloning disclosed in each.

The embryo may be cultured in vitro by any methods available to one of skill in the art. Two examples of media for culturing in the art are PZM and NCSU. Example recipes for PZM are PZM-3 and PZM-4 (108 mM NaCl, 10 mM KCl, 0.35 mM KH₂PO₄, 0.40 mM MgSO₄.7H₂O, 25.07 mM NaHCO₃, 0.20 mM Na-pyruvate, 2 mM Ca-(lactate)₂.5H₂O, 1 mM L-Glutamine, 5 mM Hypotaurine, 20 mL/L Basal Media Eagle amino acids, 10 mL/L Minimum Essential Medium nonessential amino acids, 0.05 mg/ml gentamicin, 3.00 mg/ml fatty acid-free BSA (PZM-3), 3.00 mg/ml polyvinyl alcohol (PZM-4), pH 7.3). An example recipe for NCSU is NCSU-23 ((108.73 mM NaCl, 4.78 mM KCl, 1.70 mM CaCl₂.2H₂O, 1.19 mM KH₂PO₄, 1.19 mM MgSO₄.7H₂O, 25.07 mM NaHCO₃, 1 mM L-Glutamine, 7 mM Taurine, 5 mM Hypotaurine, 0.05 mg/ml gentamicin, 4.00 mg/ml fatty acid-free BSA, pH 7.3). (Yoshioka, K. et al., Biology of Reproduction 66, 112-119 (2002).) Typically the embryos are cultured at a temperature of the average body temperature of the animal being cloned. By way of example, a pig's average body temperature varies in the range of 38.0° C. and 39.5° C., therefore the preferred temperatures for culturing porcine embryos would be in that range, with more preferred temperatures from 38.5° C. and 39.0° C. One of skill in the art may use any appropriate atmosphere for culturing the embryos. Examples are 5% CO₂ with humidified air and humidified gas consisting of 5% CO₂:5% O₂:90% N₂.

One of skill in the art will recognize that the culture may be continuous culture for a specified time period or may be the total time cultured where the culturing is interrupted by way of example by freezing the embryo. The time the embryo is not cultured is not included in the time of culturing the embryo. One of skill in the art may use any available method of freezing the embryo. The preferred animal, pig, may be frozen, for example, by delipification prior to freezing or by rapid freezing in a straw containing a microfilament inhibitor. The later method has achieved an 80% survival frequency with pig embryos. (See Dobrinsky, J. R., Reprod Suppl. 58, 325-33 (2001).)

The embryo may be transferred into the recipient female animal by any method available to one of skill in the art. The embryo may be transferred by surgical means. Various non-surgical methods of implantation are available to one of skill in the art. Non-surgical or laparoscopic implantation is more difficult in the preferred recipient animal—a pig—owing to the uterine horn. However, various methods have been developed to overcome these difficulties. Examples of devices and methods for non-surgical implantation into a pig may be found in U.S. Pat. No. 5,558,636 and U.S. Pat. No. 6,607,518, both of which are incorporated by reference in their entirety. In the preferred animal—pig—such laparoscopic or non-surgical transfer methods may be used to transfer the cloned and/or transgenic embryos into a gilt or a sow. In a preferred embodiment, the female pig is in its peak reproductive age. In another preferred embodiment, the female pig is a sow with proven maternal abilities. In certain embodiments, the transfer may be synchronized or asynchronous. A recipient maternal animal and an embryo to be transferred into the recipient are said to be “synchronized” or “synchronous” when either fertilization (for a sexually reproduced embryo, including one produced by artificial insemination) or activation (for a nuclear transfer embryo) occurs about 44 to 46 hours after the onset of standing estrus in the maternal recipient. A recipient maternal animal and an embryo to be transferred into the recipient are said to be “asynchronous” when the embryo is more developed or less developed than would be expected if the embryo and the maternal recipient were synchronized. For example, when either in vitro fertilization (for a sexually reproduced embryo) or activation (for a cloned embryo) occurs prior to the onset of standing estrus in the maternal recipient, and up to about 43 hours after the onset of standing estrus in the maternal recipient, the recipient. maternal animal and the embryo are said to be “asynchronous.”

### PREFERRED METHOD

### EXAMPLE 1

For the cloning procedure matured oocytes were obtained from Bomed (commercial supplier of porcine and cattle oocytes, Madison, Wis. Internet:www.bomed.com), a commercial supplier. Following removal of the cumulus cells, oocytes were stained with Hoechst and enucleated in the presence of cytochalasin B. Enucleated ooplast were reconstructed by placing a somatic cell (obtained via ear punch grown culture from a Duroc, a Hampshire and a terminal cross sire, respectively) into the perivitaline space and fusing the cell to the ooplast with an electrical pulse. Reconstructed oocytes were then incubated for 1-2 hrs prior to activation with an electrical pulse. Reconstructed embryos were then cultured in either PZM or NCSU at 38.5° C. for 4 days. Following four days culture reconstructed embryos were placed into a pre-equilibrated 2 ml polystyrene tube. The media used for culturing was pre-equilibrated prior to shipping. The tubes were capped, parafilmed, placed into a shipping incubator at 38° C. and shipped overnight from Austin, Tex. to Athens, Ga. Upon arrivals tubes were placed into an incubator prior to transfer. Once ready the embryos were removed from the shipping tube and placed into a 35 mm dish containing Hepes buffered M199 supplemented with 10% fetal calf serum. Embryos were then loaded into a tom cat catheter and transferred into the uterine horn of the recipient. The recipients were virgin gilts age 5-8 month old. Both natural and induced estrus recipients were used . Estrus induction was performed with prostaglandin or PMSG/hCG treatment to synchronize the recipient with the embryo. Following transfer the recipients were checked for pregnancy at 28 days and allowed to farrow. Three different cell lines (Table 1) have been tested utilizing two different media (Table 2).

TABLE 1
Number of recipients farrowing following the transfer of
porcine nuclear transfer embryos cultured for 5 days. Three
cell lines isolated from three different pigs were utilized
and all successfully produced offspring.

| Cell Line | # Recipients Transferred | # Farrowed |
|---|---|---|
| 1 | 6 | 2 (33%) |
| 2 | 4 | 3 (75%) |
| 3 | 12 | 3 (25%) |

TABLE 2
Effect of culture media on recipients farrowing

| Media | # Recipients Transferred | # Farrowed |
|---|---|---|
| NCSU | 13 | 5 (39%) |
| PZM | 8 | 3 (38%) |

### EXAMPLE 2

Embryos were reconstructed according to the methods of Example 1, using three different somatic cell lines as donors. The reconstructed embryos were either immediately transferred to a recipient or were cultured in PZM for five days prior to transfer. Embryo transfer was accomplished using the same procedure as described in Example 1. Recipients were again virgin gilts, 5-8 months, both natural and induced estrus. Following transfer, the recipients were checked for pregnancy at approximately 28 days and allowed to farrow. The number of recipients farrowing and average litter size for recipients receiving non-cultured and five-day cultured embryos are reported in Table 3.

TABLE 3
Number of recipients farrowing and average litter size
for non-cultured and five-day cultured embryos.

| # Days Embryos | # Recipients | | Avg. |
|---|---|---|---|
| Culture | Transferred | # Farrowed | Litter Size |
| 0 | 5 | 3 (60%) | 6 |
| 5 | 5 | 4 (80%) | 6 |

The five-day cultured embryo transfer results are not only comparable to those of directly transferred cloned embryos (i.e., 0 days culture) but also compare favorably with current IVF rates. That is, current IVF methods typically transfer about 25-50 blastocysts per recipient, with about 10-20% viability to term, and the average litter size being about five piglets. With five-day cultured embryos, we typically transfer about 20-30 blastocysts per recipient, with 20-30% viability to term, and an average litter size of 6 piglets per recipient.

What we claim is:

1. A method for the production of cloned piglets comprising the steps of:
(b) culturing cloned pig embryos for a period of time;
(c) transferring the cultured embryos to suitable recipients; and
(d) allowing the transferred embryos to develop to term, wherein the recipient farrowing rate is greater than 30% and the average litter size is at least 4 piglets.

2. The method of claim 1 wherein the embryos are cultured in any media capable of supporting cloned porcine embryo development prior to transfer.

3. The method of claim 1 wherein the embryos are transferred after at least 18 hours in culture.

4. The method of claim 1 wherein the embryos are transferred after more than 76 hours in culture.

5. The method of claim 1 wherein the embryos are transferred into the uterus of the recipient.

6. The method of claim 5 wherein the embryos are transferred into the uterine body of the recipient.

7. The method of claim 5 wherein the embryos are transferred into the uterine horn of the recipient.

8. The method of claim 1 in which the embryos are transferred by laparoscopy or non-surgical embryo transfer.

9. The method as claimed in claim 1 in which the embryo is transgenic.

10. The method as claimed in claim 1 wherein the embryo is frozen at least once during the culturing of the embryo.

11. The. method as claimed in claim 1 further comprising the steps of transporting the embryos to a separate facility for transfer.

12. A method for the production of cloned piglets comprising the steps of:
(a) culturing cloned pig embryos for more than 76 hours; and
(c) transferring the cultured embryos to suitable recipients by laparoscopy or non-surgical techniques; and
(d) allowing the transferred embryos to develop to term.

13. The method of claim 12 wherein the embryos are cultured in any media capable of supporting cloned porcine embryo development prior to transfer.

14. The method of claim 12 wherein the embryos are transferred into the uterus of the recipient.

15. The method of claim 14 wherein the embryos are transferred into the uterus body of the recipient.

16. The method of claim 14 wherein the embryos are transferred into the uterine horn of the recipient.

17. The method of claim 12 wherein the recipient farrowing rate is greater than 30%.

18. The method of claim 12 wherein the average litter size is at least 4 piglets.

19. The method as claimed in claim 12 in which the embryo is transgenic.

20. The method as claimed in claim 12 wherein the embryo is frozen at least once during the culturing of the embryo.

21. The method as claimed in claim 12 further comprising the steps of transporting the embryos to a separate facility for transfer.

22. A method for the production of cloned piglets comprising the steps of:
(a) culturing cloned pig embryos for at least 18 hours;
(c) transferring the cultured embryos to suitable recipients using non-surgical or laparoscopic techniques; and
(d) allowing the transferred embryos to develop to term, wherein the recipient farrowing rate is greater than 30% and the average litter size is at least 4 piglets.

23. The method of claim 22 wherein the embryos are cultured in any media capable of supporting cloned porcine embryo development prior to transfer.

24. The method of claim 22 wherein the embryos are non-surgically transferred.

25. The method of claim 22 wherein the embryos are transferred using laparoscopic techniques.
